(19) United States
(12) Patent Application Publication
(10) Pub. No.: US 2012/0309638 A1
(43) Pub. Date: Dec. 6, 2012

(54) MARKERS AND METHODS FOR DETERMINING RISK OF DISTANT RECURRENCE OF NON-SMALL CELL LUNG CANCER IN STAGE I-IIIA PATIENTS

(76) Inventors: Jacek JASSEM, Gdansk (PL); Marcin SKRZYPSKI, Sopot (PL)

(21) Appl. No.: 13/474,708

(22) Filed: May 17, 2012

(30) Foreign Application Priority Data
May 18, 2011 (PL) P.394921

(51) Int. Cl. C07H 21/02; C40B 30/04; C12Q 1/68

(52) U.S. Cl. 506/9; 536/23.1; 435/6.12

(57) ABSTRACT

New markers for determination of the high risk of NSCLC distant recurrence (distant metastases), where the markers are selected from the group of hsa.miR-192, hsa.miR-194, hsa.miR-662, hsa.miR-502.3p, hsa.miR-128 and hsa.miR-362.5p. A method for determination of the risk of distant recurrence (distant metastases) of NSCLC in surgically treated patients in stage I-IIIA after pulmonary resection, where: total RNA is isolated from a fragment of NSCLC tumor, the amount and quality of RNA in the examined sample are determined, with biotechnology methods of measuring the amount of microRNAs, preferably quantitative RT-PCR, the expression (amount) of microRNA in tumor tissue is determined; and, subsequently, the microRNA expression is analyzed according to the model of prediction of the occurrence of distant metastases, where the high expression of microRNAs: hsa.miR-192, hsa.miR-194, hsa.miR-662 and low expression of microRNAs: has.miR-502.3p, hsa.miR-128 and hsa.miR-362.5p indicate the high risk of distant metastases.

### CROSS-REFERENCE TO RELATED APPLICATIONS

This application claims foreign priority benefits to Polish Patent Application No. P.394921 filed May 18, 2011, the contents of which, including any intervening amendments thereto, are incorporated herein by reference in their entirety.

### CORRESPONDENCE ADDRESS

Inquiries from the public to applicants or assignees concerning this document should be directed to: MATTHIAS SCHOLL P.C., ATTN.: DR. MATTHIAS SCHOLL ESQ., 14781 MEMORIAL DRIVE, SUITE 1319, HOUSTON, TX 77079.

### FIELD OF THE INVENTION

The present invention relates to new markers and method of determining the risk of distant recurrence (metastases) of non-small cell lung cancer (NSCLC) in surgically treated patients who had undergone radical resection of lung tissue in stage I-IIIA of the disease.

### BACKGROUND OF THE INVENTION

Lung cancer is the most common cause of cancer-related mortality in most countries and approximately 80% of lung cancer patients are diagnosed with NSCLC.

In early stages of the disease, surgery is the treatment of choice; however, the treatment results are unsatisfactory, with about only 50% of patients being fully cured. The surgical treatment outcome is difficult to predict. Patients with the same pTNM stage and the same ‘conventional’ prognostic factors, e.g. histology, grade, age, sex and performance status, vary in their propensity to develop local or distant recurrence (distant metastases).

The most common reason of treatment failure after surgery is tumor dissemination, which invariably leads to death. Two metaanalyses that included patients from several prospective clinical trials showed that addition of chemotherapy based on platinum derivatives improves the overall survival rates of patients by approximately 5%.

Currently, the only approved selection criterion for adjuvant treatment is the stage of the disease as determined pathologically by analyzing the resected tissue. The prospective randomized studies have shown the benefit of adjuvant chemotherapy in stage II and IIIA NSCLC patients. On the other hand, treatment failure in stage I patients who are not qualified for adjuvant chemotherapy is as high as 30-40%. Given the above, there is a need to develop molecular tests that could predict the increased risk of dissemination (distant metastases) after surgery, which would allow for a more rational selection of patients to undergo adjuvant chemotherapy.

The results of research on molecular markers suggest that gene expression profiling (mRNA abundance assessment) may be used to estimate the individual risk of disease dissemination (Bhattacharjee et al. “Classification of human lung carcinomas by mRNA expression profiling reveals distinct adenocarcinoma subclasses.” Proc Natl Acad Sci USA 2001; 98: 13790-13795). A particularly useful technique for prognostication based on tumor tissue gene expression analysis is the use of reverse transcription and quantitative polymerase chain reaction (RT-PCR).

Endoh et al., in a publication entitled “Prognostic model of pulmonary adenocarcinoma by expression profiling of eight genes as determined by quantitative real-time reverse transcriptase polymerase chain reaction” (J Clin Oncol 2004; 22: 811-819), described the technique of quantification of gene expression in cancer cells.

The results of numerous studies based on this technique may ultimately lead to introduction of new molecular criteria to refine the staging of this malignancy.

From Skrzypski et al. “Three-gene expression signature predicts survival in early-stage squamous cell carcinoma of the lung.” Clin Cancer Res 2008; 14: 4794-4799 is known a gene expression profile prognostic for squamous cell lung cancer.

Concurrently to the studies on the prognostic role of gene expression (mRNA assessment), in the last few years, a new class of molecules, microRNA, has been studied for this purpose. These molecules are important in many cellular processes, such as embryogenesis, proliferation and differentiation of cells, apoptosis and oncogenesis (Earson et al. “MicroRNAs in development and disease”. Clin. Genetics 2008; 74: 296-306).

MicroRNAs are short RNA chains (19-23 nucleotides) that regulate gene expression through inhibiting mRNA translation. This effect is mediated by binding between microRNA and non-coding target sequences in mRNA molecules whereby translation in ribosomal complexes is blocked. Given the permissiveness for binding with imperfect complementarity between target sequences, one microRNA molecule may control the expression of hundreds or thousands of genes.

The prognostic potential of assessing microRNA expression in primary tumors is being investigated in many cancer types. In a publication by Yu et al. “MicroRNA signature predicts survival and relapse in lung cancer.” (Cancer Cell 2008; 13: 48-57), the authors identified a profile consisting of 5 microRNAs, which is highly predictive for prognosis in NSCLC patients in stages I-IIIA.

In the patent publication WO 2007/081720, the methods of detection, prognostication and treatment of lung cancer were revealed based on quantification of microRNA in tissue. The method of cancer detection relies on quantification of at least one microRNA in the investigated sample in reference to the control samples and resultant determination of the risk of lung cancer occurrence. Prognostication of the course of lung cancer (its propensity to recur) also consists in quantification of at least one microRNA in the investigated sample in reference to the control samples where certain values of expression are correlated to poor prognosis of lung cancer. In turn, the use of microRNA in treatment of lung cancer assumes the determination of the amount of microRNA in cancer cells in reference to control cells and application of procedures that aim to change the amount of microRNA in cancer cells.

In the Polish Patent Application No. P.388681 (corresponding to WO 2011/014083) is described a method of determination of the risk of distant metastases (and prognosis) of the NSCLC through determination of expression of at least one of the 22 microRNAs in the primary tumor of NSCLC.

From the above identified publications, the methods of establishing prognosis in stage I-IIIA NSCLC patients who underwent surgical treatment are known. These methods in principle include: (i) obtaining a sample of primary tumor tissue, (ii) RNA isolation, (iii) that is transcribed into cDNA, and (iv) establishing the amount of microRNA or mRNA in primary tumor by RT-PCR or other suitable methods known in molecular biology. Finally, the mRNA or microRNA expression value is referred to the reference expression values in a model of disease recurrence prediction, whereby certain expression values are correlated to high and certain to low risk of recurrence.

To increase the efficacy of lung cancer treatment, new and more effective methods of NSCLC recurrence risk determination are needed.

### BRIEF DESCRIPTION OF THE INVENTION

In one aspect of the invention provided are new markers for determination of the risk of distant recurrence (distant metastases) of NSCLC in surgically treated patients in stage I-IIIA after pulmonary resection, the determination comprising:
isolating RNA from primary tumor NSCLC samples;
determining the concentration and quality of RNA in the examined samples;
determining the expression (amount) of microRNA in tumor tissue using biotechniques for measuring the amount of microRNAs, preferably by quantitative RT-PCR, in the samples with cDNA; and
analyzing the microRNA expression according to the model of prediction of the occurrence of distant metastases;

wherein the new markers are selected from the group of hsa.miR-192, hsa.miR-194, hsa.miR-662, hsa miR-502.3p, hsa.miR-128, hsa.miR-362.5p, and exhibit the following levels of expression, which are indicative of a high risk of distant metastases:

| microRNA | Level of expression |
|---|---|
| hsa.miR-502.3p | Low |
| hsa.miR-192 | High |
| hsa.miR-128 | Low |
| hsa.miR-362.5p | Low |
| hsa.miR-194 | High |
| hsa.miR-662 | High |

In certain embodiments, the markers have the following sequences:

| | |
|---|---|
| hsa.miR-502.3p | AAUGCACCUGGGCAAGGAUUCA |
| hsa.miR-192 | CUGACCUAUGAAUUGACAGCC |
| hsa.miR-128 | UCACAGUGAACCGGUCUCUUUU |
| hsa.miR-362.5p | AAUCCUUGGAACCUAGGUGUGAGU |
| hsa.miR-194 | UGUAACAGCAACUCCAUGUGGA |
| hsa.miR-662 | UCCCACGUUGUGGCCCAGCAG |

In certain embodiments of the markers, the high expression of microRNA has.miR-192 is a marker of high risk of NSCLC distant recurrence (distant metastases).

In certain embodiments of the markers, the high expression of microRNA has.miR-194 is a marker of high risk of NSCLC distant recurrence (distant metastases).

In certain embodiments of the markers, the high expression of microRNA has.miR-662 is a marker of high risk of NSCLC distant recurrence (distant metastases).

In certain embodiments of the markers, the low expression of microRNA has.miR-502-3p is a marker of high risk of NSCLC distant recurrence (distant metastases).

In certain embodiments of the markers, the low expression of microRNA has.miR-128 is a marker of high risk of NSCLC distant recurrence (distant metastases).

In certain embodiments of the markers, the low expression of microRNA has.miR-362-5p is a marker of high risk of NSCLC distant recurrence (distant metastases).

In certain embodiments of the invention, markers take the form of the risk index (RI) that is linearly correlated with the risk of distant recurrence (distant metastases) and is expressed in the general formula of:

RI=microRNA A+microRNA B+microRNA C . . . +microRNA N

where the expressions (microRNA A . . . N) take the number format, with the preferred values of “0” or “1”, where the value of “0” indicates low risk of distant metastases and the value of “1” indicates high risk of distant metastases for a specific microRNA, where these values are derived from models of prediction of disease recurrence for microRNAs listed in Table 1, and where (RI) denotes the sum of expressions, the values of which are derived from the expression of individual microRNAs (at least 2 and at most 6 microRNAs) and where high risk of recurrence is inferred if the RI value for a given patient is higher than the RI threshold value, determined in studies on larger patient cohorts; however, this threshold value is contained within 10-90% of the RI value for the entire population of the NSCLC stage I-IIIA patients.

In certain embodiments of the invention, provided is a kit comprising a set of probes, at least one of each hybridizes with at least a part of or with the entire microRNAs sequence being examined, or corresponding cDNA sequences, which enables the analysis of the amount of the abovementioned microRNAs in the NSCLC tumor sample under investigation, and preferably includes reagents, such as buffers, enzymes and other chemicals necessary to determine the amount of the investigated microRNA in a given sample.

A method of determining the risk of distant recurrence (distant metastases) of NSCLC in surgically treated patients in stage I-IIIA after pulmonary resection, the method comprising
isolating RNA from primary tumor NSCLC samples;
determining the concentration and quality of RNA in the examined samples;
determining the expression (amount) of microRNA in tumor tissue using biotechniques for measuring the amount of microRNAs, preferably by quantitative RT-PCR in the samples with cDNA; and
analyzing the microRNA expression according to the model of prediction of the occurrence of distant metastases;

wherein high levels of expression (number of copies) of microRNA selected from the group consisting of hsa.miR-192, hsa.miR-194, hsa.miR-662, and low levels of expression of microRNA selected from the group consisting of hsa miR-502.3p, hsa.miR-128, hsa.miR-362.5p, are indicative of a high risk of recurrence (distant metastases).

In certain embodiments of the method, the high expression of microRNA has.miR-192 is a marker of high risk of NSCLC distant recurrence (distant metastases).

In certain embodiments of the method, the high expression of microRNA has.miR-194 is a marker of high risk of NSCLC distant recurrence (distant metastases).

In certain embodiments of the method, the high expression of microRNA has.miR-662 is a marker of high risk of NSCLC distant recurrence (distant metastases).

In certain embodiments of the method, the low expression of microRNA has.miR-502-3p is a marker of high risk of NSCLC distant recurrence (distant metastases).

In certain embodiments of the method, the low expression of microRNA has.miR-128 is a marker of high risk of NSCLC distant recurrence (distant metastases).

In certain embodiments of the method, the low expression of microRNA has.miR-362-5p is a marker of high risk of NSCLC distant recurrence (distant metastases).

In certain embodiments of the method, the markers take the form of the risk index (RI) that is linearly correlated with the risk of distant recurrence (distant metastases) and is expressed in the general formula of:

RI=microRNA A+microRNA B+microRNA C . . . +microRNA N

where the expressions (microRNA A . . . N) take the number format, with the preferred values of “0” or “1”, where the value of “0” indicates low risk of distant metastases and the value of “1” indicates high risk of distant metastases for a specific microRNA, where these values are derived from models of prediction of disease recurrence for microRNAs listed in Table 1, and where (RI) denotes the sum of expressions, the values of which are derived from the expression of individual microRNAs (at least 2 and at most 6 microRNAs) and where high risk of recurrence is inferred if the RI value for a given patient is higher than the RI threshold value, determined in studies on larger patient cohorts; however, this threshold value is contained within 10-90% of the RI value for the entire population of the NSCLC stage I-IIIA patients.

In certain embodiments of the method, provided is a kit comprising a set of probes, at least one of each hybridizes with at least a part of or with the entire microRNAs sequence being examined, or corresponding cDNA sequences, which enables the analysis of the amount of the abovementioned microRNAs in the NSCLC tumor sample under investigation, and preferably includes reagents, such as buffers, enzymes and other chemicals necessary to determine the amount of the investigated microRNA in a given sample.

### DETAILED DESCRIPTION OF THE INVENTION

Unexpectedly the new markers allow for a significantly more precise determination of the risk of distant metastases and prognosis in stage I-IIIA NSCLC.

This invention is based on the assessment of the expression of at least one out of 6 microRNAs listed in Table 1, the measured value of which is referred to the reference expression values in a model in which certain values are correlated to the high or low risk of distant metastases after surgical treatment, respectively. The marker sensu stricto of high risk of recurrence is either high or low expression of respective microRNAs. Importantly, the highest prognostic information (preferred embodiment) is obtained from the simultaneous analysis of expression of several (combinations of 2 up to 6) markers (high or low microRNAs expression). In this way, the final marker is concluded where the marker is the sum of the prognostic information derived from the result of expression of at least 2 and at most 6 microRNAs that are described in this application.

The method comprises the acquisition of a sample of primary tumor tissue (frozen tissue or tissue fixed in formalin and stored in parafin) in which microRNA expression is measured. The tissue material is obtained during routine surgical procedure, which does not impose any risk on patients.

The microRNA expression analysis may be carried out with the use of various molecular techniques, e.g. quantitative RT-PCR, oligonucleotide microarrays or bead array technologies (Illumina®).

Identification of the markers and their analysis, which reaches beyond currently used diagnostic tests, will facilitate the assessment of the risk of recurrence.

TABLE 1

| | | | MicroRNA |
|---|---|---|---|
| | | | expression |
| | | | indicative |
| | | | of high |
| | | | risk of |
| | | | distant |
| | microRNA | Sequences microRNA | metastases |
| 1 | hsa.miR-502.3p | AAUGCACCUGGGCAAGGAUUCA | LOW |
| 2 | hsa.miR-192 | CUGACCUAUGAAUUGACAGCC | HIGH |
| 3 | hsa.miR-128 | UCACAGUGAACCGGUCUCUUUU | LOW |
| 4 | hsa.miR-362-5p | AAUCCUUGGAACCUAGGUGU | LOW |
| | | GAGU | |
| 5 | hsa.miR-194 | UGUAACAGCAACUCCAUGUGGA | HIGH |
| 6 | hsa.miR-662 | UCCCACGUUGUGGCCCAGCAG | HIGH |

New markers for the determination of the risk of distant recurrence (distant metastases) of NSCLC in surgically treated patients in stage I-IIIA after pulmonary resection where new markers are selected from the group of microRNAs: hsa.miR-192, hsa.miR-194, hsa.miR-662, has.miR-502.3p, hsa.miR-128, hsa.miR-362.5p and if their measured expression is as shown in Table 1, then it indicates the high risk of distant recurrence. The new markers have the sequences shown in Table 1.

New markers according to the invention where the high expression of microRNA has.miR-192 is a marker of high risk of NSCLC distant recurrence (distant metastases).

New markers according to the invention where the high expression of microRNA has.miR-194 is a marker of high risk of NSCLC distant recurrence (distant metastases).

New markers according to the invention where the high expression of microRNA has.miR-662 is a marker of high risk of NSCLC distant recurrence (distant metastases).

New markers according to the invention where the low expression of microRNA has.miR-502-3p is a marker of high risk of NSCLC distant recurrence (distant metastases).

New markers according to the invention where the low expression of microRNA has.miR-128 is a marker of high risk of NSCLC distant recurrence (distant metastases).

New markers according to the invention where the low expression of microRNA has.miR-362-5p is a marker of high risk of NSCLC distant recurrence (distant metastases).

New markers according to the invention where markers take the form of the risk index (RI) that is linearly correlated with the risk of distant recurrence (distant metastases) and is expressed in the general formula of:

RI=microRNA A+microRNA B+microRNA C . . . +microRNA N

where the expressions (microRNA A . . . N) take the number format, with the preferred values of “0” or “1”, where the value of “0” indicates low risk of distant metastases and the value of “1” indicates high risk of distant metastases for a specific microRNA, where these values are derived from models of prediction of disease recurrence for microRNAs and where (RI) denotes the sum of expressions, the values of which are derived from the expression of individual microRNAs (at least 2 and at most 6 microRNAs) and where high risk of recurrence is inferred if the RI value for a given patient is higher than the RI threshold value, determined in studies on larger patient cohorts; however, this threshold value is contained within 10-90% of the RI value for the entire population of the NSCLC stage I-IIIA operable patients.

The invention also comprises the method of determination of the risk of distant recurrence (distant metastases) of NSCLC in surgically treated patients in stage I-IIIA after pulmonary resection, comprising: isolating RNA from primary tumor NSCLC samples; determining the concentration and quality of RNA in the examined samples; determining the expression (amount) of microRNA in tumor tissue using biotechniques for measuring the amount of microRNAs in the samples, preferably by quantitative RT-PCR; and analyzing the microRNA expression according to the model of prediction of the occurrence of distant metastases; wherein high levels of expression (number of copies) of microRNA selected from the group consisting of hsa.miR-192, hsa.miR-194, hsa.miR-662, and low levels of expression of microRNA selected from the group consisting of hsa miR-502.3p, hsa.miR-128, hsa.miR-362.5p, are indicative of a high risk of recurrence (distant metastases). Preferably, at least 2 and at most 6 microRNAs are analyzed simultaneously, and in such cases the marker takes the form of a Risk Index (RI), i.e. the sum of prognostic information inferred from each microRNA included in a given Risk Index.

The method according to the invention where the high expression of microRNA has.miR-192 is a marker of high risk of NSCLC distant recurrence (distant metastases).

The method according to the invention where the high expression of microRNA has.miR-194 is a marker of high risk of NSCLC distant recurrence (distant metastases).

The method according to the invention where the high expression of microRNA has.miR-662 is a marker of high risk of NSCLC distant recurrence (distant metastases).

The method according to the invention where the low expression of microRNA has.miR-502-3p is a marker of high risk of NSCLC distant recurrence (distant metastases).

The method according to the invention where the low expression of microRNA has.miR-128 is a marker of high risk of NSCLC distant recurrence (distant metastases).

The method according to the invention where the low expression of microRNA has.miR-362-5p is a marker of high risk of NSCLC distant recurrence (distant metastases).

The method according to the invention where markers take the form of the risk index (RI) that is linearly correlated with the risk of distant recurrence (distant metastases) and is expressed in the general formula of:

RS=microRNA A+microRNA B+microRNA C . . . +microRNA N

where, the expressions (microRNA A . . . N) take the number format, with the preferred values of “0” or “1”, where the value of “0” indicates low risk of distant metastases and the value of “1” indicates high risk of distant metastases for a specific microRNA, where these values are derived from models of prediction of disease recurrence for microRNAs and where (RI) denotes the sum of expressions, the values of which are derived from the expression of individual microRNAs (at least 2 and at most 6 microRNAs) and where high risk of recurrence is inferred if the RI value for a given patient is higher than the RI threshold value, to be determined in studies on larger patient cohorts; however, this threshold value is contained within 10-90% of the RI value for the entire population of the NSCLC stage I-IIIA operable patients.

For the new markers and the method for risk determination used is a kit comprising a set of probes, at least one of each hybridizes with at least a part of or with the entire microRNAs sequence being examined, or corresponding cDNA sequences, which enables the analysis of the amount of the abovementioned microRNAs in the NSCLC tumor sample under investigation, and preferably includes reagents, such as buffers, enzymes and other chemicals necessary to determine the amount of the investigated microRNA in a given sample.

### EXAMPLES

The invention is further explained by the following examples:

### Example 1

Total RNA containing a fraction of markers (microRNA) from Table 1 was isolated from tumor tissue with miRNeasy Mini Kit (50) (Qiagen) Catalog No. 217004.

The concentration and quality of RNA was assessed with RNA Lab Chip (Bianalyzer Agilent 2100).

Subsequently, RNA was retrotranscribed to cDNA (RT reaction) with TaqMan MicroRNA RT kit Cat No 4366596 (Applied Biosystems) containing specific stem-loop primers specific to microRNAs—MegaPlex RT Cat. no. 4401091 (Applied Biosystems) in accordance with the manufacturer's recommendations.

MicroRNA (cDNA resultant from RT reaction) was quantified with quantitative PCR reaction using specific primers pairs and fluorescent TaqMan probes and polymerase with 5′ nuclease activity in microfluidic cards—TaqMan Low Density Arrays Part Number 4400238 (Applied Biosystems) in HT 7900 cycler (Applied Biosystems) with reaction conditions in accordance with manufacturer's recommendations (Applied Biosystems).

Raw expression results (Ct values) were obtained through SDS.2.1 (Applied Biosystems) software and the expression was normalized against the geometric mean of expression of U6 RNA and RNU48 RNA.

The values of expression of microRNA listed in Table 1 are correlated with the reference expression values in a model of risk prediction (distant metastases prediction) wherein the defined values correspond to high risk of recurrence (distant metastases).

The model of risk prediction was determined for each of the 6 microRNAs listed in Table 1. For each microRNA, an expression threshold was found for accuracy of distant metastases prediction to be the highest.

If the expression of the microRNA is either above or below the determined threshold, respectively (see Table 2 for each microRNA), then that indicates the high risk of disease recurrence.

Also in Table 2, the results of the comparison of the probability of metastases-free survival (MFS) for two groups of patients: (i) with low risk of distant metastases according to the markers 1-6 and (ii) with high risk of distant metastases according to the markers 1-6 are presented. In all cases, the difference in MFS between two groups was statistically significantly different (p<0.05).

TABLE 2

| | | | Expression | |
|---|---|---|---|---|
| | | | indicating | Statistical |
| | | | high risk | significance |
| | microRNA | microRNA sequence | of recurrence | p-value |
| 1 | hsa.miR-502.3p | AAUGCACCUGGGCAAGGAUUCA | Low | 0.0095 |
| 2 | hsa.miR-192 | CUGACCUAUGAAUUGACAGCC | High | 0.0195 |
| 3 | hsa.miR-128 | UCACAGUGAACCGGUCUCUUUU | Low | 0.0318 |
| 4 | hsa.miR-362.5p | AAUCCUUGGAACCUAGGUGUGAGU | Low | 0.0344 |
| 5 | hsa.miR-194 | UGUAACAGCAACUCCAUGUGGA | High | 0.0401 |
| 6 | hsa.miR-662 | UCCCACGUUGUGGCCCAGCAG | High | 0.049 |

### Example 2

The preferred embodiment of the invention is the marker that is constructed through averaging of the prognostic effect of expression of the individual microRNAs from Table 1 (markers 1-6). The description of this invention embodiment follows below.

The measurement of the expression of microRNA was carried out in samples of the primary tumors, collected from 51 stage I and II patients with squamous cell lung cancer who had either a disease recurrence (distant metastases) (n=21) or were free of disease after a median of observation of 5.7 years (n=30).

In this example, freshly frozen tissue material was used for microRNA isolation. This kind of material is obtained routinely during surgery and its collection has no impact on the surgical treatment of the patients.

Total RNA containing a fraction of markers (microRNA) from Table 1 was isolated from tumor tissue with miRNeasy Mini Kit (50) (Qiagen) Catalog No. 217004.

The concentration and quality of RNA were assessed with RNA Lab Chip (Bianalyzer Agilent 2100).

Subsequently, RNA was retrotranscribed to cDNA (RT reaction) with TaqMan MicroRNA RT kit Cat No 4366596 (Applied Biosystems) containing specific stem-loop primers specific to microRNAs—MegaPlex RT Cat. no. 4401091 (Applied Biosystems) in accordance with the manufacturer's recommendations.

MicroRNA (cDNA resultant from RT reaction) was quantified with quantitative PCR reaction using specific primers pairs and fluorescent TaqMan probes and polymerase with 5′ nuclease activity in microfluidic cards—TaqMan Low Density Arrays Part Number 4400238 (Applied Biosystems) in HT 7900 cycler (Applied Biosystems) with reaction conditions in accordance with manufacturers' recommendations (Applied Biosystems).

Raw expression results (Ct values) were obtained through SDS.2.1 (Applied Biosystems) software and in the case of this experiment the expression was normalized against the expression of U6 RNA and RNU48 RNA.

The values of expression of microRNA listed in Table 1 were referred to the reference expression values in models of recurrence (distant metastases) risk prediction where defined values correspond to high risk of recurrence.

The final marker of the high risk of recurrence described in this example included 6 microRNAs. For each microRNA, an expression threshold was found to obtain the highest accuracy of distant metastases prediction. For microRNAs: hsa.miR-192, hsa.miR-194, hsa.miR-662, the value of ‘0’ or ‘-1’ was assigned if the expression was lower or higher than the corresponding threshold values, respectively. For microRNAs: miR-502.3p, hsa.miR-128, hsa.miR-362.5p, the value of ‘0’ or ‘1’ was assigned if the expression was lower or higher than the corresponding threshold values, respectively. For each patient, the partial Risk Index values inferred from the expression of the six microRNAs were added according to the formula below and the Risk Index was calculated for all the analyzed patients. Next, 51 patients were assigned to the low risk or high risk groups if their Risk Index values were either higher or lower than the median value for the Risk Index for the entire cohort.

Risk Index=microRNA 1+microRNA 2+microRNA 3 . . . +microRNA 6

TABLE 3

| | | | The expression | The resultant |
|---|---|---|---|---|
| | | Threshold | indicating the | partial value of |
| | microRNA | value | high risk | the Risk Index |
| 1 | hsa-miR-502-3p | 1.1 | low | 0 |
| 2 | hsa-miR-192 | 0.7 | high | −1 |
| 3 | hsa-miR-128 | 2.31 | low | 0 |
| 4 | hsa-miR-362-5p | 0.74 | low | 0 |
| 5 | hsa-miR-194 | 0.98 | high | −1 |
| 6 | hsa-miR-662 | 7.5 | high | −1 |

### BRIEF DESCRIPTION OF THE DRAWINGS

FIG. 1 represents the metastases-free survival (MFS) curves for the low and high risk patient groups according to the Risk Index comprising six microRNAs. The label “High” denotes high Risk Index values that correspond to the low risk of distant metastases and longer MFS. The label “Low” denotes low Risk Index values that correspond to the high risk of distant metastases and shorter MFS. The difference of MFS between these two groups was highly statistically significant (p<0.001).

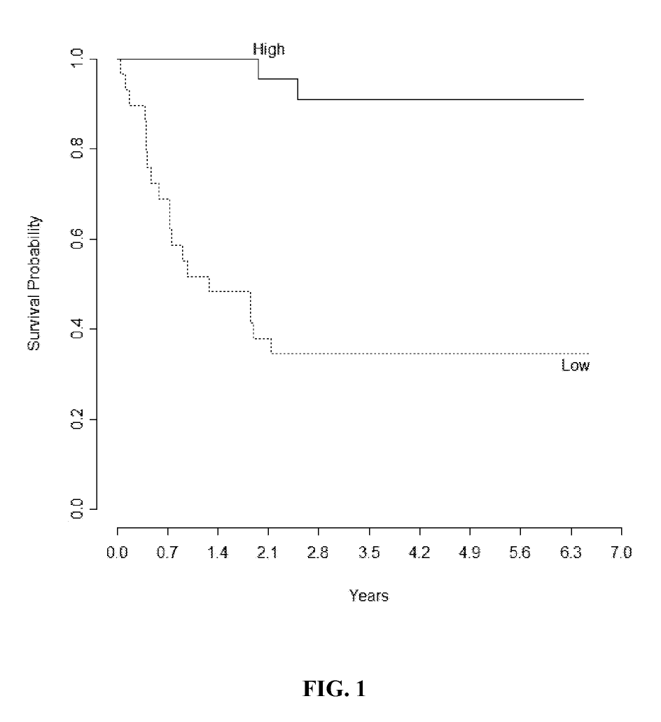

1. New markers for determination of the risk of distant recurrence (distant metastases) of NSCLC in surgically treated patients in stage I-IIIA after pulmonary resection, the determination comprising:
isolating RNA from primary tumor NSCLC samples;
determining the concentration and quality of RNA in the examined samples;
determining the expression (amount) of microRNA in tumor tissue using biotechniques for measuring the amount of microRNAs, preferably by quantitative RT-PCR in the samples with cDNA; and
analyzing the microRNA expression according to the model of prediction of the occurrence of distant metastases;
distinct in that the new markers are selected from the group of hsa.miR-192, hsa.miR-194, hsa.miR-662, hsa miR-502.3p, hsa.miR-128, hsa.miR-362.5p, and exhibit the following levels of expression, which are indicative of the high risk of distant metastases:

| | Expression level indicating the |
|---|---|
| microRNA | high risk of distant metastases |
| hsa.miR-502.3p | Low |
| hsa.miR-192 | High |
| hsa.miR-128 | Low |
| hsa.miR-362.5p | Low |
| hsa.miR-194 | High |
| hsa.miR-662 | High. |

2. The new markers of claim 1, distinct in that the markers have the following sequences:

| | |
|---|---|
| hsa.miR-502.3p | AAUGCACCUGGGCAAGGAUUCA |
| hsa.miR-192 | CUGACCUAUGAAUUGACAGCC |
| hsa.miR-128 | UCACAGUGAACCGGUCUCUUUU |
| hsa.miR-362.5p | AAUCCUUGGAACCUAGGUGUGAGU |
| hsa.miR-194 | UGUAACAGCAACUCCAUGUGGA |
| hsa.miR-662 | UCCCACGUUGUGGCCCAGCAG. |

3. The new markers of claim 1, distinct in that high expression of microRNA miR-192 is a marker of high risk of distant recurrence of NSCLC (distant metastases).

4. The new markers of claim 1, distinct in that high expression of microRNA miR-194 is a marker of high risk of distant recurrence of NSCLC (distant metastases).

5. The new markers of claim 1, distinct in that high expression of microRNA miR-662 is a marker of high risk of distant recurrence of NSCLC (distant metastases).

6. The new markers of claim 1, distinct in that low expression of microRNA miR-502-3p is a marker of high risk of distant recurrence of NSCLC (distant metastases).

7. The new markers of claim 1, distinct in that low expression of microRNA miR-128 is a marker of high risk of distant recurrence of NSCLC (distant metastases).

8. The new markers of claim 1, distinct in that low expression of microRNA miR-362-5p is a marker of high risk of distant recurrence of NSCLC (distant metastases).

9. The new markers of claim 1, distinct in that each marker in the form of a Risk Index (RI) is linearly correlated with the risk of distant recurrence (distant metastases) and is expressed by the formula:
RI=microRNA A+microRNA B+microRNA C . . . +microRNA N
where the expressions (microRNA A . . . N) take the number format, in the preferred values of “0” or “1”, where the value of “0” indicates low risk of distant metastases and the value of “1” indicates high risk of distant metastases in a model of prediction of disease recurrence for each microRNA as in claim 1, and where (RI) denotes the sum of expressions, the values of which are derived from the expression of individual microRNAs (at least 2 and at most 6 microRNAs), and where high risk of recurrence is concluded if the RI value for a given patient is higher than the RI threshold value, determined in studies on larger patient cohorts; however, this threshold value is contained within 10-90% of the RI values for the entire population of the NSCLC stage I-IIIA patients.

10. The new markers of claim 1, distinct in that a kit to perform the microRNA expression analysis is used, and the kit comprises a set of probes, at least one of each hybridizes with at least a part of or with the entire microRNAs sequence being examined, or corresponding cDNA sequences, which enables the analysis of the amount of the abovementioned microRNAs in the NSCLC tumor sample under investigation, and preferably includes reagents, such as buffers, enzymes and other chemicals necessary to determine the amount of the investigated microRNA in a given sample.

11. The new markers of claim 2, distinct in that a kit to perform the microRNA expression analysis is used, and the kit comprises a set of probes, at least one of each hybridizes with at least a part of or with the entire microRNAs sequence being examined, or corresponding cDNA sequences, which enables the analysis of the amount of the abovementioned microRNAs in the NSCLC tumor sample under investigation, and preferably includes reagents, such as buffers, enzymes and other chemicals necessary to determine the amount of the investigated microRNA in a given sample.

12. A method of determining the risk of distant recurrence (distant metastases) of NSCLC in surgically treated patients in stage I-IIIA after pulmonary resection, the method comprising:
isolating RNA from primary tumor NSCLC samples;
determining the concentration and quality of RNA in the examined samples;
determining the expression (amount) of microRNA in tumor tissue using biotechniques for measuring the amount of microRNAs, preferably by quantitative RT-PCR in the samples with cDNA; and
analyzing the microRNA expression according to the model of prediction of the occurrence of distant metastases;
distinct in that high levels of expression (number of copies) of microRNAs selected from the group consisting of hsa.miR-192, hsa.miR-194, hsa.miR-662, and low levels of expression of microRNAs selected from the group consisting of hsa miR-502.3p, hsa.miR-128, hsa.miR-362.5p, are indicative of a high risk of recurrence (distant metastases).

13. The method of claim 12, distinct in that high expression of microRNA miR-192 is a marker of high risk of distant recurrence of NSCLC (distant metastases).

14. The method of claim 12, distinct in that high expression of microRNA miR-194 is a marker of high risk of distant recurrence of NSCLC (distant metastases).

15. The method of claim 12, distinct in that high expression of microRNA miR-662 is a marker of high risk of distant recurrence of NSCLC (distant metastases).

16. The method of claim 12, distinct in that low expression of microRNA miR-502-3p is a marker of high risk of distant recurrence of NSCLC (distant metastases).

17. The method of claim 12, distinct in that low expression of microRNA miR-128 is a marker of high risk of distant recurrence of NSCLC (distant metastases).

18. The method of claim 12, distinct in that low expression of microRNA miR-362-5p is a marker of high risk of distant recurrence of NSCLC (distant metastases).

19. The method of claim 12, distinct in that each marker is in the form of a Risk Index (RI) of distant metastases is linearly correlated with the risk of distant recurrence (distant metastases) and is expressed in the general formula of:
RI=microRNA A+microRNA B+microRNA C . . . +microRNA N
where the expressions (microRNA A . . . N) take the number format, in the preferred values of “0” or “1”, where the value of “0” indicates low risk of distant metastases and the value of “1” indicates high risk of distant metastases in a model of prediction of disease recurrence for each microRNA as in claim 1 and where (RI) denotes the sum of expressions, the values of which are derived from the expression of individual microRNAs (at least 2 and at most 6 microRNAs), and where high risk of recurrence is concluded if the RI value for a given patient is higher than the threshold value, to be determined in studies on larger patient cohorts; however, this threshold value is contained within 10-90% of the RI value for the entire population of the NSCLC stage I-IIIA patients.

20. A method of claim 12, distinct in that a kit to perform the microRNA expression analysis is used, and the kit comprises a set of probes, at least one of each hybridizes with at least a part of or with the entire microRNAs sequence being examined, or corresponding cDNA sequences, which enables the analysis of the amount of the abovementioned microRNAs in the NSCLC tumor sample under investigation, and preferably includes reagents, such as buffers, enzymes and other chemicals necessary to determine the amount of the investigated microRNA in a given sample.
